SEQUENCE LISTING (2 sequences)

SEQ ID NO: 1 - DNA, 1157 bp, Artificial Sequence
  recombinant
atgcatgcgaattcgtcgacggatcctcctaggactagtgggcccatgccgggtcagaaccagatcggcgactacaagctcatcgagctggccggggagggcagtttcggcaaagtgtggaaggcgcggcgcgcgggcagcctgcagacggtggccgtcaagctcatcaccaagcacggcaagaacgacaaggacctgcgctcgctccgccaggagattgagatcctgcgcaagctgcagcacccaaacatcatcgccatgctggacgcctttgagaccaagaacgacttctgcgtggtcaccgagtttgcgcagggcgagctgttccacatcttggaggacgaccggtgcctgcccgagggggtggtgcgcagcgtggggcgccagctggtgcaggcccttcactacctgcacaccaaccgcatcatccaccgggacatgaagccccaaaacattctcatcagcgccaacggcgccgtcaagctgtgcgactttggctttgcgcgcctgatgagcagcaacactctggtggtgacgtcgatcaagggcaccccgctgtacatggcgccggagctggtgcaggagcagccctacaaccacacggtggacctctggtccctgggcgtcatcctgtacgagctgtttgtgggacagccgcccttctacaccacctccatctacaccctcattaagcagatcgtgcgggagccagtcaagttccccgacggcatgtcgccgaccttcacctccttcctgcaggcgcgcctcacctccttccttcctttcttgcttcctttcttgcgtgctttctttccttgcgccttcttcgttttcccgtccacccagcgggtccggtgcctgcccagggctgctttgtgcatgggctgccgggtgctggccgcctgctagccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcgcccgggatgcatgc

SEQ ID NO: 2 - protein, 302 aa, Artificial Sequence
  recombinant
YTAEKEIGKGSFATVYRGHLTSDKSQHVAIKEVSRAKLKNKKLLENLEIEIAILKKIKHPHIVGLIDCERTSTDFYLIMEYCALGDLTFLLKRRKELMENHPLLRTVFEKYPPPSENHNGLHRAFVLSYLQQLASALKFLRSKNLVHRDIKPQNLLLSTPLIGYHDSKSFHELGFVGIYNLPILKIADFGFARFLPNTSLAETLCGSPLYMAPEILNYQKYNAKADLWSVGTVVFEMCCGTPPFRASNHLELFKKIKRANDVITFPSYCNIEPELKELICSLLTFDPAQRIGFEEFFANKVV

(19) United States
(12) Patent Application Publication
(10) Pub. No.: US 2016/0369307 A1
(43) Pub. Date: Dec. 22, 2016

(54) METHOD OF INCREASING BIOMASS AND LIPID CONTENT IN A MICRO-ORGANISM AND A GENETICALLY MODIFIED MICRO-ORGANISM EXHIBITING ENHANCED AUTOPHAGY

(71) Applicant: RELIANCE INDUSTRIES LIMITED, Maharashtra (IN)

(72) Inventors: Gautam DAS, Andhra Pradesh (IN); Santanu DASGUPTA, Mumbai (IN); Raja KUMAR, Navi Mumbai (IN); Venkatesh PRASAD, Karnataka (IN); Nanjappa DEEPAK, Bangalore (IN); Niraja SONI, Navi Mumbai (IN); Amol DATE, Sanpada (IN); Badrish Ranjitlal SONI, Gujarat (IN); Pasupuleti NAGARJUNA, Andhra Pradesh (IN)

(73) Assignee: RELIANCE INDUSTRIES LIMITED, Maharashtra (IN)

(21) Appl. No.: 15/110,683

(22) PCT Filed: Jan. 9, 2015
(86) PCT No.: PCT/IN2015/000011

§ 371 Date: Jul. 8, 2016

(30) Foreign Application Priority Data
Jan. 10, 2014 (IN) 2316/MUM/2013

(51) Int. Cl. C12P 7/64; C07K 14/405; C12N 1/12; C12N 15/82

(52) U.S. Cl. CPC C12P 7/64; C12N 15/8247; C07K 14/405; C12N 1/12

(57) ABSTRACT

According to an embodiment of the invention, there is provided a method of increasing biomass and lipid content in a micro-organism comprising cloning in a vector an exogenous gene sequence selected from the group comprising Atg1 gene, Atg6 gene, and Atg8 gene sequence wherein the sequence is codon optimized for said micro-organism, for inducing autophagy; introducing the vector containing the gene into the genome of the micro-organism to yield a genetically modified micro-organism; and growing the genetically modified micro-organism in suitable medium. According to another embodiment of the invention there is provided a method of increasing biomass and lipid content in a micro-organism exposed to stress, comprising treating the microorganism with an autophagy inducing agent.

### TITLE OF THE INVENTION

Method of increasing biomass and lipid content in a micro-organism and a genetically modified micro-organism exhibiting enhanced autophagy

### FIELD OF THE INVENTION

This invention relates to a method of increasing biomass and lipid content in a micro-organism and a genetically modified micro-organism exhibiting enhanced autophagy.

### BACKGROUND OF THE INVENTION

During stressful conditions, different organisms subject themselves to intracellular degradation by various means to combat stress and promote survival for normal cell function. In eukaryotes, mainly two modes exist for intracellular degradation—the proteasome degradation pathway and autophagy. In the former pathway, protein complexes called proteasomes function to enzymatically break-down unnecessary or damaged proteins. Autophagy, on the other hand, is the route used, to break-down cytoplasmic materials, including organelles, and therefore yields diverse degradation products. Other techniques of stress resistance in eukaryotes generally include distinct genetic modifications to deal with individual stress factors like adverse changes in pH, temperature etc. But, autophagy is a mode by which organisms may deal with varied stresses simultaneously and cumulatively.

Specifically, autophagy is a catabolic process that mediates turnover of intracellular constituents, specifically, defective constituents, of a cell and plays a vital role in cellular growth, survival and homeostasis. Autophagy is initiated by the formation of an isolation membrane that expands to engulf a portion of the cytoplasm to form an autophagosome which then fuses with a lysosome to form an autolysosome. The material captured within the autolysosome and the inner membrane are then degraded by enzymes such as lysosomal hydrolases.

Organisms use varied techniques for stress resistance including genetic modification addressing stress factors such as changes in pH, temperature, oxygen/nitrogen/carbon dioxide levels, salinity, availability of sunlight or water, exposure to ultraviolet (UV) radiation etc. However, such techniques for combating stress function to eliminate the effect of individual specific stress factors. The autophagy pathway, on the other hand, has potential to cumulatively eliminate the effects of numerous stress factors through a single coordinated process.

Autophagy is a catabolic process that adjusts cellular biomass and function in response to diverse stimuli and stress factors like starvation and infection to enable a cell to survive in a hostile environment. Autophagy thus involves nutrient recycling within a cell for the purpose of combating stress.

Various organisms such as plants, algae and the like possess the cellular machinery to engage in autophagy. A recent study shows the presence of autophagy genes in Chlorella (Jiang et al., Analysis of autophagy genes in microalgae: Chlorella as a potential model to study mechanism of autophagy (2012). Eukaryotic microalgae possess several unique metabolic attributes of relevance to biofuel production, including the accumulation of significant quantities of triacylglycerol; the synthesis of storage starch (amylopectin and amylose), which is similar to that found in higher plants; and the ability to efficiently couple photosynthetic electron transport to H₂ production (Radakovits, R., et al., Genetic engineering of algae for enhanced biofuel production; Eukaryotic Cell Vol 9, 486-501 (2010)).

However, the need of modulating, and specifically enhancing a cell's productivity, modulation of autophagy for growth, survival and combating stress conditions in a more effective manner is not completely understood. Specifically, mechanisms for modulating autophagy in a target organism are yet to be understood for organisms that have potential of being used for various industrial applications. Suitable examples of such organisms include, but are not limited to, photosynthetic organisms. Accordingly, there exists a need for an efficient method for enhancing autophagy in organisms for growth and combating stress, and organisms modified at genetic level for exhibiting enhanced autophagy in order to achieve high yield of products of interest from such organisms.

### SUMMARY OF THE INVENTION

According to an embodiment of the invention, there is provided a method of increasing biomass and lipid content in a micro-organism comprising:
a. cloning in a vector an exogenous gene sequence selected from the group comprising Atg 1 gene, Atg6 gene, and Atg8 gene sequence wherein the sequence is at least 50% homologous with Atg 1 gene, Atg6 gene, and Atg8 gene, codon optimized for said micro-organism, for inducing autophagy;
b. introducing the vector containing the gene into the genome of the micro-organism to yield a genetically modified micro-organism; and
c. growing the genetically modified micro-organism in suitable medium.

According to another embodiment of the invention there is provided a method of increasing biomass and lipid content in a micro-organism exposed to stress, comprising treating the micro-organism with an autophagy inducing agent.

According to yet another embodiment of the invention there is provided a genetically modified micro-organism exhibiting enhanced autophagy, the micro-organism comprising a vector carrying an exogenous gene sequence selected from the group comprising Atg1 gene, Atg6 gene, and Atg8 gene sequence wherein the sequence is at least 50% homologous with Atg1 gene, Atg6 gene, and Atg8 gene codon optimized for said micro-organism, known to induce autophagy. One of the genetically modified micro-organisms prepared according to an embodiment of the invention, namely Chlamydomonas reinhardtii CC 125, has been deposited on 18^{th} Dec. 2014 at Culture Collection of Algae and Protozoa (CCAP), SAMS Limited, Scottish Marine Institute, Dunbeg, Oban, Argyll, PA37 1QA, UK and has CCAP Accession Number CCAP 11/171.

According to another embodiment of the invention there is provided a genetically modified eukaryotic micro-organism exhibiting enhanced autophagy comprising a nucleic acid sequence of SEQ ID No. 1

According to yet another embodiment of the invention there is provided a genetically modified micro-organism exhibiting enhanced autophagy comprising a nucleic acid sequence coding a protein kinase domain of SEQ ID No. 2.

According to still another embodiment of the invention there is provided a vector comprising a regulatory nucleic acid segment operably coupled to a nucleic acid sequence of SEQ ID No. 1.

According to yet another embodiment of the invention there is provided a vector comprising a regulatory nucleic acid segment operably coupled to a nucleic acid sequence encoding a polypeptide comprising an amino acid sequence of SEQ ID No. 2.

According to still another embodiment of the invention there is provided a nucleic acid sequence comprising SEQ ID No. 1.

According to yet another embodiment of the invention there is provided a nucleic acid sequence encoding a polypeptide comprising an amino acid sequence of SEQ ID No. 2

According to still another embodiment of the invention there is provided a polypeptide comprising an amino acid sequence of SEQ ID No. 2

### BRIEF DESCRIPTION OF THE DRAWINGS

FIG. 1A is a comparison of Control plates and the z-vad-fmk treated plates after 13 days of UV exposure.

FIG. 1B graphically represents the optical density at 750nm of the Control cultures versus the optical density of the cultures treated with z-vad-fmk on the 13^{th} day after UV exposure.

FIG. 2 shows the Control plates and in duplicate the LiCl treated plates after 4 days of salinity stress exposure.

FIG. 3 is a graphical comparison of LysoTracker Mean Fluorescence Intensity (MFI) after UV treatment at 30minutes and 2 days versus an untreated sample.

FIG. 4A and FIG. 4B show flow cytometer analysis data after UV exposure followed by 30 minutes of recovery and 2 days of recovery respectively.

FIG. 5 is a vector map of pChlamy_1 with ATG1 cloned using Kpnl and Nde I.

FIG. 6 is a colony PCR (Polymerase Chain Reaction) image confirming the Atg 1 (autophagy protein) transformants in Chlamydomonas reinhardtii.

FIG. 7A and FIG. 7B are western blot films showing bands at ≅75KDa and ≅13KDa respectively indicating elevated levels of Atg8 protein in transformants 3 and 5.

FIG. 8A is a graphical comparison of the percentage of Chlorophyll positive cells in the UV treated samples analyzed in FACS at Day 2, Day 4, Day 6, Day 8 and Day 10 post UV exposure.

FIG. 8B is a graphical comparison of the percentage of Chlorophyll positive cells in the untreated samples analyzed in FACS at Day 2, Day 4, Day 6, Day 8 and Day 10.

FIG. 8C is a Nile Red assay of samples for which MFI was checked 4 days post-UV treatment.

FIG. 8D is a comparison of the lysosomal activity in Wild Types and transformants.

FIG. 9A is a graphical comparison of Chlorophyll a auto fluorescence of untreated Wild type versus the Transformants.

FIG. 9B is a graphical comparison of Chlorophyll a auto fluorescence of UV treated Wild type versus the Transformants.

FIG. 9C is a graphical comparison of OD at 750 nm of untreated Wild type and Transformants.

FIG. 9D is a graphical comparison of OD at 750 nm of UV treated Wild type and Transformants.

FIG. 10A and FIG. 10B graphically show that the transformants have a clear advantage over wild type in salinity stress tolerance.

FIG. 11A and FIG. 11B graphically show that the transformants have a clear advantage over wild type in temperature stress tolerance.

FIG. 12A graphically compares the growth advantage of Transformant 5 over Wild Types under salinity stress.

FIG. 12B graphically compares the growth advantage of Transformant 5 over Wild Types under high temperature stress.

FIG. 12C graphically compares the growth advantage of Transformant 5 over Wild Types under high light stress.

### DETAILED DESCRIPTION OF THE PREFERRED EMBODIMENTS

For simplicity and illustrative purposes, the present invention is described by referring mainly to exemplary embodiments thereof. In the following description, numerous specific details are set forth in order to provide a thorough understanding of the present invention. It will be apparent, however, to one of ordinary skill in the art that the present invention may be practiced without limitation to these specific details. In other instances, well known methods/techniques have not been described in detail so as not to unnecessarily obscure the present invention.

Throughout this specification the word “comprise”, or variations such as “comprises” or “comprising”, will be understood to imply the inclusion of a stated element, or step, or group of elements, or steps, but not the exclusion of any other element, or step, or group of elements, or steps.

The use of the expression “at least” or “at least one” suggests the use of one or more elements or ingredients or quantities, as the use may be in the embodiment of the disclosure to achieve one or more of the desired objects or results.

Optionally, the genetically modified micro-organism prepared according to an embodiment of the invention is exposed to abiotic stresses like ultraviolet radiation (UV), salinity, light, unfavourable temperature, alkalinity, nutrient limitation, oxidative stress, senescence, sulfur deficiency, carbon deficiency, nitrogen use inefficiency, stress due to biotic reasons like virus, bacteria, fungus or other stress causing pathogens. Optionally, the genetically modified micro-organism prepared according to an embodiment of the invention is also chemically treated with LiCl to further induce autophagy.

Autophagy can be induced in the micro-organism either by genetic modification or by chemical induction of autophagy or a combination of these.

Preferably, the method of increasing biomass and lipid content in a micro-organism involves the use of the vector pChlamy_1. Optionally, the exogenous gene has at least 52% homology with Atg1 gene of yeast. Optionally, the exogenous gene having at least 52% homology with Atg1 gene of yeast is obtained from Chlorella.

Preferably, the micro-organism is a photosynthetic micro-organism.

The stress can be abiotic stresses like ultraviolet radiation (UV), salinity, light, unfavourable temperature, alkalinity, nutrient limitation, oxidative stress, senescence, sulfur deficiency, carbon deficiency, nitrogen use inefficiency, stress due to biotic reasons like virus, bacteria, fungus or other stress causing pathogens. Preferably, when the stress is UV, the UV exposure is not more than 6 hours. Preferably, the autophagy inducing agent is z-vad-fmk when the stress is UV. Still more preferably, the micro-organism is treated with 1 mM to 1M of z-vad-fmk for 1 minute to 5 days. Preferably, the micro-organism is kept in the dark for 24 hours after UV exposure followed by exposure to light.

Preferably, when the stress is salinity, the salinity exposure is not more than 10 days. Preferably, the autophagy inducing agent is LiCl when the stress is salinity.

In a preferred embodiment of the invention, a genetically modified photosynthetic micro-organism exhibiting enhanced autophagy comprises the vector pChlamy_1. Preferably, the exogenous gene carried by the vector has at least 52% homology with Atg1 gene of yeast. Preferably, the exogenous gene having at least 52% homology with Atg1 gene of yeast is obtained from Chlorella.

According to an embodiment of the invention there is provided a method for inducing enhanced autophagy in an organism by genetically engineering the organism to exhibit enhanced autophagy during stress conditions and yield one or more products of interest. Preferably, the autophagy is measured by flow cytometry.

Preferably, the products of interest are biofuel and, high value chemicals. In a preferred embodiment of the invention, at least one endogenous autophagy gene is over-expressed in the organism so as to result in enhanced autophagy. In yet another preferred embodiment of the invention, at least one exogenous autophagy gene is introduced into the genetic material of the organism and is over-expressed so as to result in enhanced autophagy. The endogenous or exogenous autophagy gene is preferably an algal autophagy gene. More preferably, the over-expression is achieved through genetic manipulation for the expression of the Atg 1 gene or recombinant derivatives thereof Alternatively, the over-expression is achieved through genetic manipulation for the expression of the Atg6 gene or recombinant derivatives thereof In another embodiment, the exogenous autophagy gene may be a naturally occurring gene and/or derivatives thereof from the same or other organisms.

More preferably, the photosynthetic micro-organism transformed by genetic manipulation is an alga, still more preferably, Chlamydomonas and Chlorella. Conventionally practiced methods for genetic engineering in a particular organism may be employed for over-expressing the autophagy genes in said organism. The autophagy gene may be cloned in appropriate DNA carriers (such as vectors) for transformation and expression in the organism, and the stable transformants may be analysed by conventional analysis techniques including but not limited to Polymerase Chain Reaction (PCR) or Southern Blotting, and finally, the genetically modified organism may be screened by electron microscopy or other reporter-based or biochemical approaches such as marker genes. Preferably, the reporter assay involves the use of Atg8 protein cleavage method that can be used either by fusion to a green fluorescent protein (GFP) or by antibody-based techniques.

Alternatively, lysotracker assays can be used.

Preferably the autophagy referred to in the present invention comprises, but is not limited to mitophagy, and ribophagy. In another embodiment the autophagy may be selective autophagy.

Typically, the stress may be, but not limited to, environmental and artificial stress. Typically the type of stress may be, but not limited to, slight and mild stress sufficient to trigger autophagy.

According to another aspect of the invention there is provided a genetically modified micro-organism exhibiting enhanced autophagy, comprising at least one autophagy gene. Preferably, the gene is Atg1 or Atg6 or recombinant derivatives thereof. The gene may be exogenously introduced or endogenous genes may be genetically engineered for overexpression thereof. Optionally, at least one gene regulating autophagy is over-expressed. In a preferred embodiment of the invention, the organism is a eukaryotic micro-organism. Preferably, the eukaryotic organism is an algae and more preferably, Chlamydomonas and Chlorella. Algae has been used for the production of biodiesel and high value chemicals by biotechnological manipulations, unrelated to autophagy. Therefore, presently, modulating autophagy and obtaining the desired biodiesel and high value chemicals from algae are of high interest.

According to another embodiment of the invention there is provided a genetic construct for expressing enhanced autophagy in eukaryotes, the construct comprising at least one genetically modified autophagy gene. Preferably, the genetic construct further comprises at least one of a promoter, an enhancer, an activator and a termination sequence. Preferably, the autophagy gene is Atg1 or Atg6. Such autophagy gene may be a naturally occurring gene and/or derivatives thereof from the same or other organisms. Preferably, the promoter is a viral promoter, more preferably Chlorella viral promoter. Preferably the enhancer is obtained from a plant source. The genetic construct may be cloned using a DNA carrier, including but not limited to a viral carrier and a non-viral carrier such as plasmids. Further, for the purpose of this description, one or more such genetic constructs may be integrated into the genome of the target organism.

According to another embodiment of the invention, there is provided a genetic construct for expressing enhanced autophagy in eukaryotes, the construct comprising at least one genetically modified gene regulatory sequence. Preferably, the regulatory sequence is a promoter or an enhancer. Preferably, the enhancer is obtained from a plant source. The genetically modified gene regulatory sequence is configured for overexpression of at least one autophagy gene. Such autophagy gene may be a naturally occurring gene and/or derivatives thereof from the same or other organisms.

The genetic construct may be cloned using a DNA carrier, including but not limited to a viral carrier and a non-viral carrier such as plasmids. Further, for the purpose of this description, one or more such genetic constructs may be integrated into the genome of the target organism.

Preferably the products of interest are biofuel and, high value chemicals. In a preferred embodiment of the invention, at least one endogenous autophagy gene is over-expressed in the organism so as to result in enhanced autophagy. In yet another preferred embodiment of the invention, at least one exogenous autophagy gene is introduced into the genetic material of the organism and is over-expressed so as to result in enhanced autophagy. The endogenous or exogenous autophagy gene is preferably an algal autophagy gene. More preferably, the over-expression is achieved through genetic manipulation of the Atg1 gene or Atg6 gene or recombinant derivatives thereof. More preferably, the organism transformed by genetic manipulation is an alga, still more preferably, Chlamydomonas and Chlorella. Conventionally practiced methods for genetic engineering in a particular organism may be employed for over-expressing the autophagy genes in said organism. The autophagy gene may be cloned in appropriate vectors for expression in the micro-organism and the stable transformants may be analysed by conventional analysis techniques including but not limited to Polymerase Chain Reaction (PCR) or Southern Blotting, and finally, the genetically modified micro-organism may be screened by electron microscopy or other reporter-based or biochemical approaches such as marker genes. Preferably, the reporter assay involves the use of Atg8 protein cleavage method that can be used either by fusion to a green fluorescent protein (GFP) or by antibody-based techniques.

According to still another embodiment of the invention, there is provided a method for producing one or more products of interest from genetically modified micro-organisms as described herein above. Preferably, the products of interest are biofuel and high value chemicals. More preferably the high value chemicals include, but are not limited to, pharmaceuticals, omega fatty acids and nutraceuticals.

The genetically modified micro-organisms are prepared according to an embodiment of the invention and are then subjected to specific environmental stresses to modulate the expression of the exogenous/endogenous genes for regulating autophagy in response to the stress such as the biotic or abiotic stress, and further evaluated for their increased tolerance to the said stress and production of chemicals including but not limited to high value chemicals therefrom.

A general overview of the steps involved in preparing the genetically modified micro-organism according to an embodiment of the invention is as follows:
1) Isolate naturally occurring micro-organism such as algae;
2) Design appropriate vectors for molecular cloning;
3) Cloning of autophagy gene in appropriate vector;
4) Transformation of appropriate vector containing the autophagy gene in algae; and
5) Screening of transformants for presence of over-expressed autophagy gene.

Microorganisms are genetically engineered by using DNA carriers including but not limited to plasmids to integrate into the organism's genome a construct which over-expresses the autophagy gene. The selection of positive transformants is done using molecular techniques like PCR or. Southern Blotting.

It is to be understood that the foregoing general description of the present embodiments of the invention is intended to provide an overview or framework for understanding the nature and character of the invention.

Any discussion of documents, acts, materials, and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

In order that those skilled in the art will be better able to practice the present disclosure, the following examples are given by way of illustration and not by way of limitation.

### EXAMPLE 1

1×10⁶ cells/ml of Chlorella sorokiniana were grown for three days in Tris-Acetate-Phosphate (TAP) medium and then exposed to UV at 250000 μJ/cm² for one minute using CL-1000 UV crosslinker. Immediately after UV exposure, 25 μM Z-vad-fmk was added to the TAP medium. The cultures were kept in the dark for 24 hours. Cultures were then exposed to light of 2000 lux for 12 hours followed by 12 hours of darkness. Initially the cultures were bleached and then only z-vad-fmk treated cultures were seen to revive after 10 days.

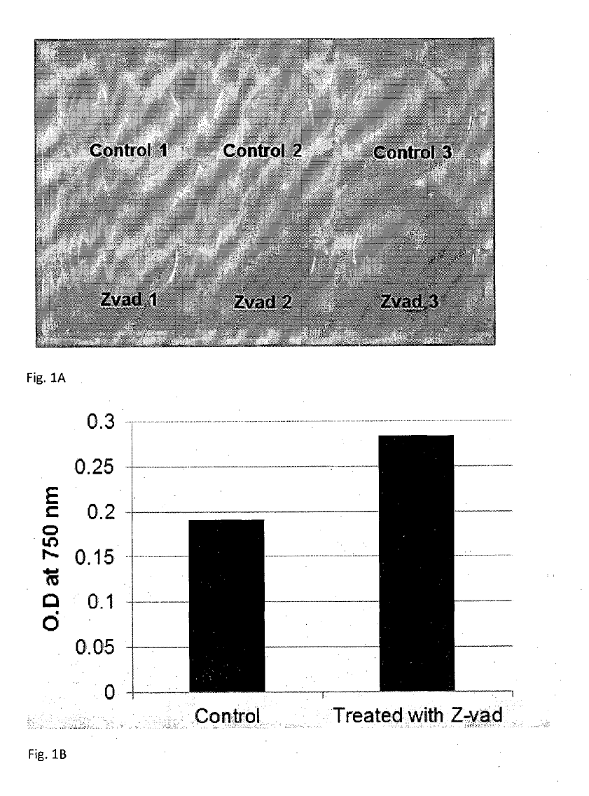

FIG. 1A shows the Control plates and the z-vad-fmk treated plates in triplicate after 13 days of UV exposure. It is clear that only the z-vad-fmk treated plates show viable cultures. FIG. 1B graphically represents the optical density at 750 nm of the Control cultures versus the optical density of the cultures treated with z-vad-fmk on the 13^{th} day after UV exposure. From these figures it is clear that z-vad-fmk induced autophagy in the Chlorella sorokiniana cells helping the cells to recover from UV exposure. Autophagy is chemically induced by z-vad-fmk in microalgae which prevents cell death in cultures exposed to UV. The pale Control plates show that cells exposed to UV die in the absence of z-vad-fmk which appears to be inducing autophagy in the z-vad-fmk treated cells. Thus, it can be concluded that even in environmental stress conditions like exposure to UV, treatment of the algal cells with z-vad-fmk prevents cell death, increases the biomass and lipid content of the algal cells, and concomitantly yields an increased amount of commercially valuable products like oils which are known to be produced by the algal cells.

### EXAMPLE 2

2×10⁶ cells/ml of Chlorella sorokiniana were grown for three days in Tris-Acetate-Phosphate (TAP) medium and then stress was induced in the cells by adding 1.2% salinity to the medium and growing the cells in the said medium for 4 days. 5mM LiCl was added to the TAP medium in the Experimental plates, but not in the Control. Cell counts were done at Day 0 i.e. the day on which cultures were exposed to 1.2% salinity stress and on Day 4 thereafter as shown in the Table 1 below:

TABLE 1

| | Cell Density at Day 0 | Cell Density at Day 4 |
|---|---|---|
| Plate type | (cells/ml) | (cells/ml) |
| Control | 2.6 × 10⁶ | 5.38 × 10⁶ |
| Experimental plate 1 | 2.6 × 10⁶ | 6.57 × 10⁶ |
| containing TAP + 0.6% | | |
| Salinity + 5 mM LiCl | | |
| Experimental plate 2 | 2.6 × 10⁶ | 7.22 × 10⁶ |
| containing TAP + 1.2% | | |
| Salinity + 5 mM LiCl | | |

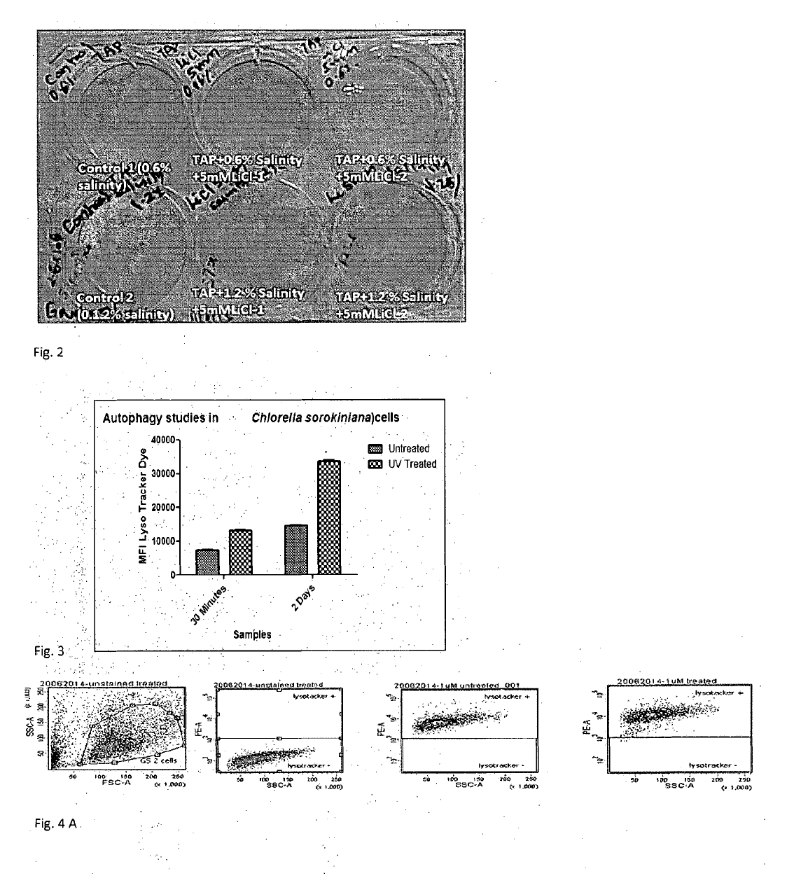

FIG. 2 shows the Control plates and in duplicate the LiCl treated plates after 4 days of salinity stress exposure. It is clear that the LiCl treated plates show higher cell counts implying greater cell viability than the Control. It is clear that autophagy is chemically induced by LiCl in microalgae which increases cell growth in cultures exposed to salinity stress. Thus, it can be concluded that even in environmental stress conditions like exposure to salinity, treatment of the algal cells with LiCl prevents cll death, increases the biomass and lipid content of the algal cells, and concomitantly yields an increased amount of commercially valuable products like oils which are known to be produced by the algal cells.

### EXAMPLE 3

2×10⁶ cells/ml of Chlorella sorokiniana were grown for three days in Tris-Acetate-Phosphate (TAP) medium and then exposed to UV at 250000 μJ/cm² for one minute using CL-1000 UV crosslinker. Recovery of the cells was checked at 30 minutes after which the cells were kept in the dark for 24 hours, and then cultures were then exposed to light of 2000 lux for 12 hours followed by 12 hours of darkness. Recovery was checked at 2 days post-exposure using Fluorescence Activated Cell Sorting (FACS), Phycoerythrin Channel. LysoTracker Red dye (1μM) was used to stain the lysosomes in the cells for five minutes at room temperature and then Mean Fluorescence Intensity (MFI) of the cells was measured and compared against MFI of cells of the same age and strain type which were not exposed to UV. The fluorescent labelled cells were analysed in Phycoerythrin (PE) Channel using BD FACS ARIA III flow cytometer. (Fluorescent Probe used: LysoTracker RED DND-99; Ex/Em: 577/590 nm). FIG. 3 shows this comparison graphically, from which it is clear that after UV treatment, MFI of the dye taken up by the cells doubled which in turn indicates increase in the number of lysosomes due to increased autophagic activity of the cells.

As is clear from FIG. 3, there is an increase in LysoTracker Mean Fluorescence Intensity (MFI) after UV treatment and the increase is more pronounced 2 days after UV treatment. FIG. 4A and FIG. 4B show flow cytometer analysis data after UV exposure followed by 30 minutes of recovery and 2 days of recovery respectively. Flow cytometry labels and tracks acidic organelles like Lysosomes in live cells. Lysosome increase in number when autophagy is induced in a cell. Hence, it is clear that UV treatment given as per the method described above results in an increase in autophagy in cells.

### EXAMPLE 4

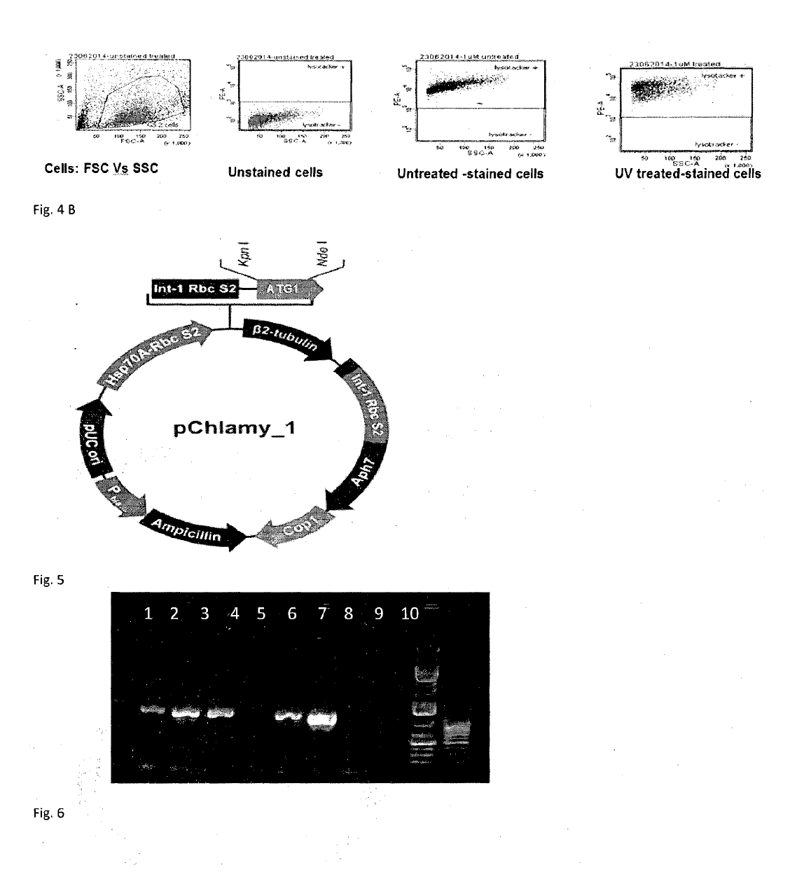

Cells of Chlamydomonas were genetically modified to induce autophagy in them by the following steps:
1. Standard computational methods were used to identify a gene in the Chlorella variabilis genome, which has 52% homology with Atg1 gene of yeast i.e. Saccharomyces cerevesiae. The said gene in Chlorella is herein referred to as the “Chlorella Atg1 gene”.
2. The Chlorella Atg 1 gene, which is understood to hypothetically yield a protein named CHLNCDRAFT_26970, was synthesised using known methods of gene synthesis.
3. Then, the Chlorella Atg 1 gene was inserted into the KpnI and NdeI restriction sites of Life Technologies' pChlamy_1 vector by known methods, at the site as shown in FIG. 5.
4. E.coli cells were transformed with the recombinant plasmid and the transformed cell cultures were expanded using known techniques. Thereafter, E. coli DNA plasmids were isolated and linearized.
5. The recombinant linearised plasmids were then used to transform Chlamydomonas using electroporation and selected on hygromycin-containing medium. PCR was used to confirm the Atg1 (autophagy protein) transformants in Chlamydomonas reinhardtii as shown in FIG. 6. In FIG. 6, wells 1 to 5 correspond to colonies 1 to 5, of which 4 showed bands after staining. So, it can be concluded that four colonies out of five analysed have been confirmed to be transformed and contain the Atg1 gene as Atg1 gene-specific primers we used to verify the same. In FIG. 6, well 6 is the positive control, well 7 is the no template control, well 8 corresponds to PCR with the wild type organism, well 9 shows a 1kb ladder while well 10 shows a 100bp ladder.
6. Gene sequencing of the nucleic acid construct containing the Chlorella Atg1 gene was done and the gene sequence obtained is shown in the sequence listing SEQ ID No. 1.

Western blotting was used to characterize the expression of proteins. Total soluble protein was extracted and SDS-PAGE migrated. The protein was electro-transferred on PVDF membrane. Western blotting was performed using primary antibody namely Anti-Atg8 and secondary antibody namely Goat Anti-Rabbit IgG H&L (HRP) preadsorbed. Detection on Photographic Film was done by ECL Kit.

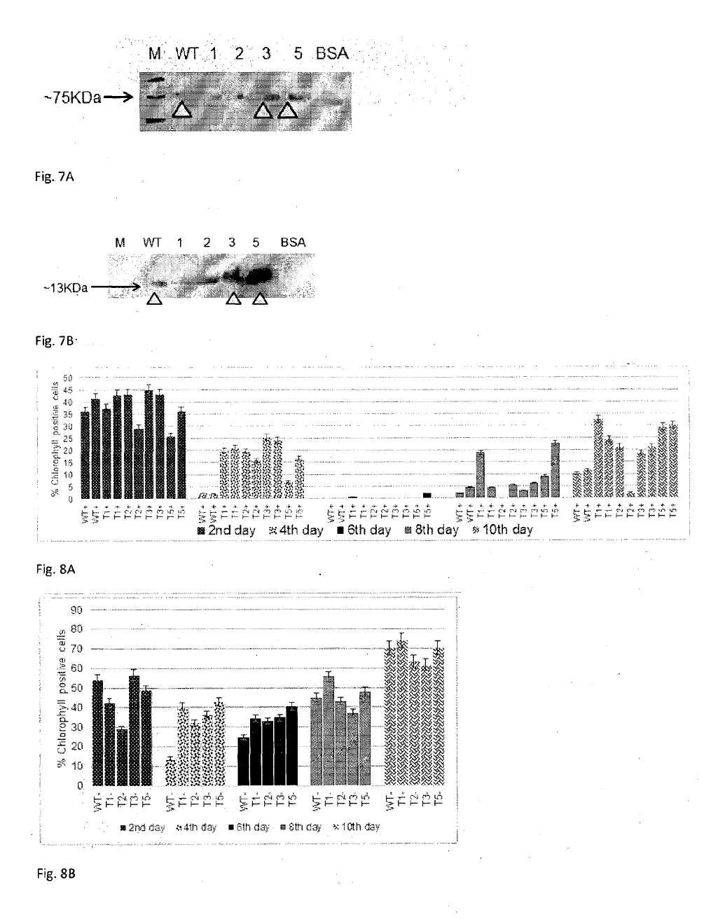

If the expression of the Atg1 is high, Atg8 also shows elevated levels of expression. The western blot detected two bands at ≅75 KDa and ≅13 KDa as shown in FIG. 7A and 7B respectively. Transformant 3 (corresponding to well 3 of PCR FIG. 6) and Transformant 5 (corresponding to well 5 of PCR FIG. 6) showed elevated levels of Atg8 protein compared to wild type. This confirmed the presence of the Atg1 transformants in Chlamydomonas using anti-Atg8 antibody.

### EXAMPLE 5

The genetically modified Chlamydomonas reinhardtii cells (i.e. Transformants 1, 2, 3 and 5 corresponding to the wells 1, 2, 3 and 5 of PCR FIG. 6) of Example 4 and separately the Wild Type strain were then exposed to UV at 250000 μJ/cm² for one minute using CL-1000 UV crosslinker followed by 24 hours of keeping the cells in the dark, and then recovery was checked at various points of time post-exposure using Fluorescence Activated Cell Sorting (FACS), Phycoerythrin Channel. LysoTracker Red dye (1μM) was used to stain the lysosomes in the cells for five minutes at room temperature and then Mean Fluorescence Intensity (MFI) of the cells was measured and compared against MFI of cells of the same age and strain type which were not exposed to UV and cells which were genetically modified as per Example 4 prior to UV exposure. The fluorescent labelled cells were analysed in Phycoerythrin (PE) Channel using BD FACS ARIA III flow cytometer. (Fluorescent Probe used: LysoTracker RED DND-99; Ex/Em: 577/590 nm).

On Day 3 post UV exposure it was found that the cells started to undergo chlorosis. On Day 7 post UV exposure it was found that almost all the cells were bleached. On Day 14 post UV exposure it was found that the transformants recovered better than the Wild Type strain.

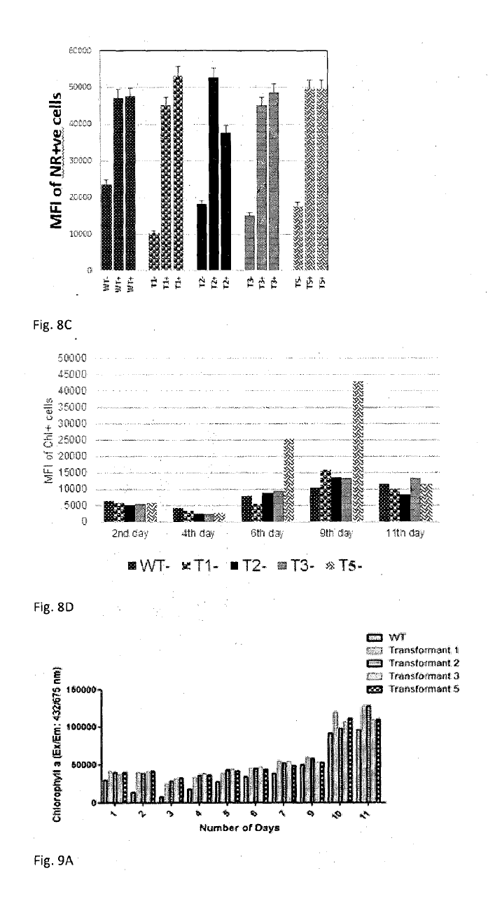

FIG. 8A is a graphical comparison of the percentage of Chlorophyll positive cells in the UV treated samples analyzed in FACS at Day 2, Day 4, Day 6, Day 8 and Day 10 post UV exposure. FIG. 8B is a graphical comparison of the percentage of Chlorophyll positive cells in the untreated samples analyzed in FACS at Day 2, Day 4, Day 6, Day 8 and Day 10. FIG. 8C is a Nile Red assay of samples for which MFI was checked 4 days post-UV treatment while FIG. 8D is a comparison of the lysosomal activity in Wild Types and transformants. In FIGS. 8A, 8B, 8C and 8D, WT=Wild Type; T=Transformant; −=No UV treatment; +=UV treated. From 8A, 8B, 8C and 8D, it is clear that UV treated transformants had higher MFI and therefore showed more autophagic activity as compared to UV treated Wild type cells, while untreated transformants also had higher MFI and therefore showed more autophagic activity as compared to untreated Wild type cells. Also, Wild type UV treated cells showed higher MFI as compared to Wild type untreated cells, as also UV treated transformants showed higher MFI as compared to untreated transformants. Particularly, the higher MFI for the transformants in FIG. 8C, where nile red assay was done to determine lipid quantity also indicates an increased lipid content in the transformants. Thus, the transformants not only have increased biomass, but also have increased lipid content.

### EXAMPLE 6

1×10⁷ cells/Int of genetically modified Chlamydomonas reinhardtii (i.e. Transformants 1, 2, 3 and 5 corresponding to the wells 1, 2, 3 and 5 of PCR FIG. 6) of Example 4 and 1×10⁷ cells/ml separately the Wild Type strain were exposed to UV at 250000 μJ/cm² using CL-1000 UV crosslinker for one minute followed by 24 hours of keeping the cells in the dark. Cultures were then exposed to light of 2000 lux for 12 hours followed by 12 hours of darkness. Then recovery was checked daily post-exposure by measuring Optical Density at 750 nm and Chlorophyll-a auto fluorescence at Ex/Em:432/675 nm in Tecan plate reader.

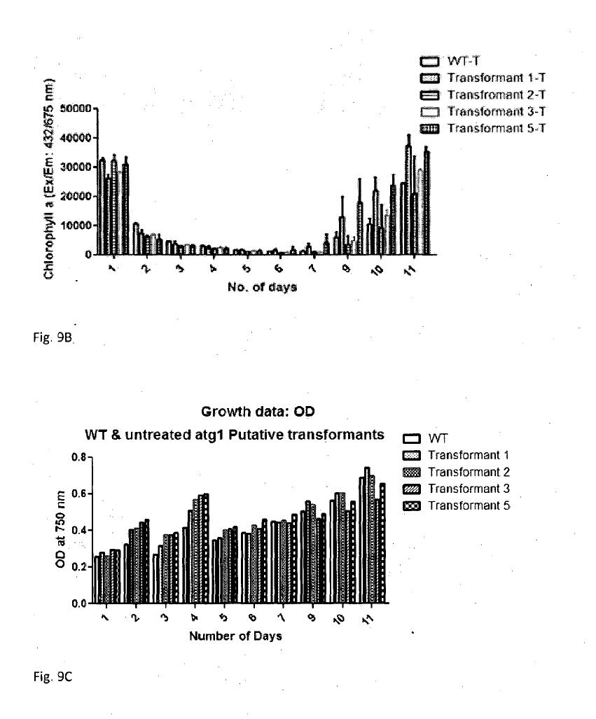

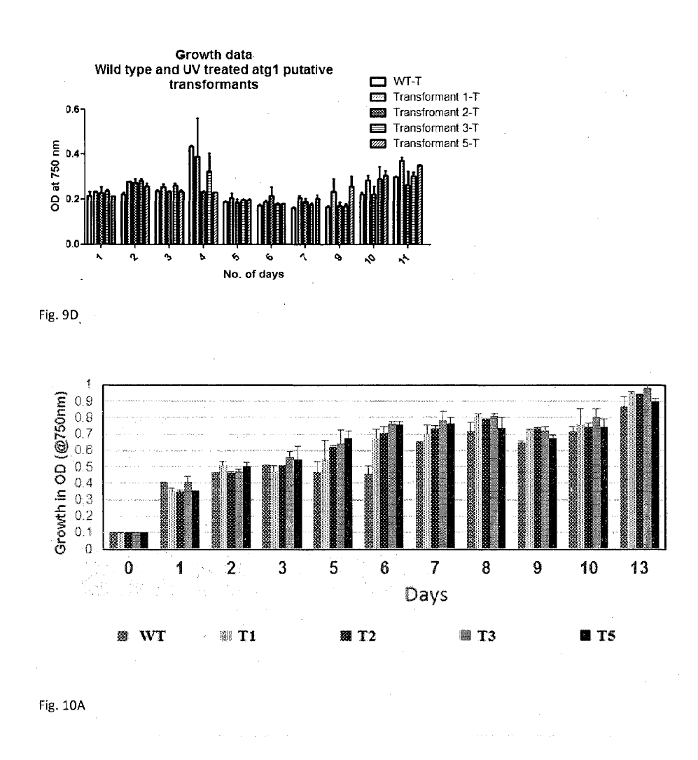

FIG. 9A is a graphical comparison of Chlorophyll a auto fluorescence of untreated Wild type versus the Transformants while FIG. 9B is a graphical comparison of Chlorophyll a auto fluorescence of UV treated Wild type versus the Transformants. After UV exposure, cultures were almost bleached. It is clear that from 7th day onwards Atg1-Transformants no. 1, 3, 5 recovered better compared to the Wild Type, both in UV treated and in untreated samples studied. FIG. 9C is a graphical comparison of OD at 750 nm of untreated Wild type and Transformants while FIG. 9D is a graphical comparison of OD at 750 nm of UV treated Wild type and Transformants.

From the above figures it is clear that in untreated samples, the growth of Transformants was better or almost comparable to wild type. In UV treated samples, from the 9^{th} day onwards OD of Transformants 1, 3 and 5 was higher than Wild type. Hence, after UV stress, Atg1-Transformants recover better than wild type.

### EXAMPLE 7

The genetically modified Chlamydomonas reinhardtii cells (i.e. Transformants 1, 2, 3 and 5 corresponding to the wells 1, 2, 3 and 5 of PCR FIG. 6) of Example 4 were subjected to salinity stress i.e. 2% salinity. It was found that 4 days after salinity stress removal, Transformants recover when transferred to normal conditions while WT almost fail to recover. From FIG. 10A and FIG. 10B it is clear that the transformants have a clear advantage over wild type in salinity stress tolerance.

### EXAMPLE 8

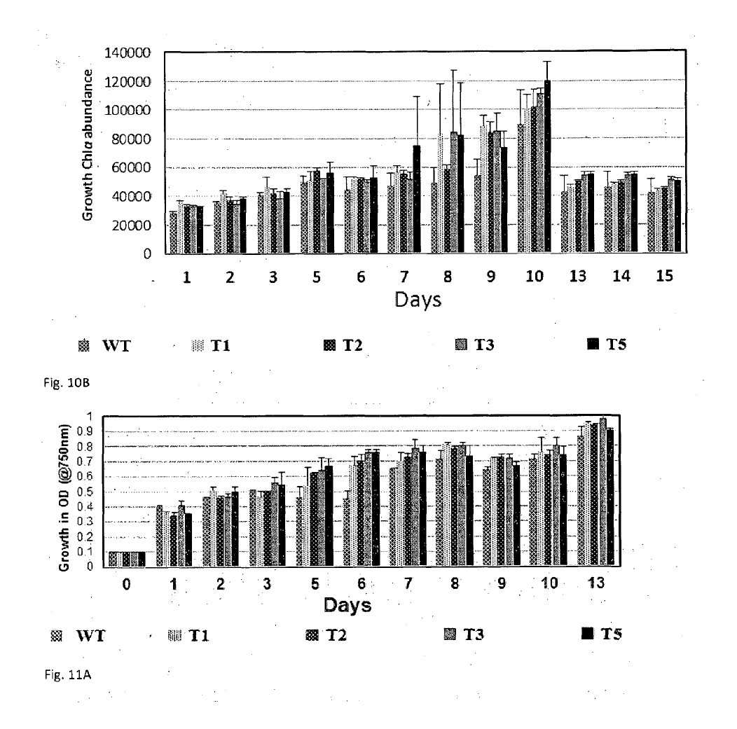

The genetically modified Chlamydomonas reinhardtii cells (i.e. Transformants 1, 2, 3 and 5 corresponding to the wells 1, 2, 3 and 5 of PCR FIG. 6) of Example 4 were subjected to temperature stress i.e. temperature of 37° C. It was found that 6 days after continuous exposure to the temperature stress, Transformants grow better at higher temperature while Wild Types look pale. From FIG. 11A and FIG. 11B it is clear that the transformants have a clear advantage over wild type in temperature stress tolerance.

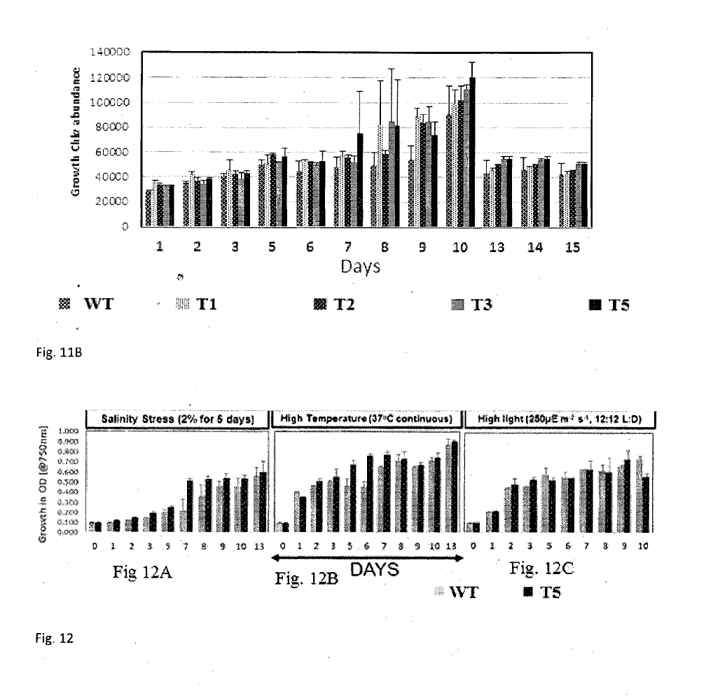

In conclusion, FIG. 12A, FIG. 12B and FIG. 12C graphically compare the growth advantage of Transformant 5 over Wild Types under different stresses namely salinity stress, high temperature stress and high light stress respectively. From FIG. 12A it is evident that Transformants take approximately 3 days less time to recover from salinity stress and reach stationary phase of growth cycle. Further, Transformants take approximately 3 days less time to reach stationary phase of growth cycle at high temperature as per FIG. 12B. Also, high light stress showed very little difference in the growth of the cultures as per FIG. 12C.

From the above Examples, it is clear that autophagy can be induced in microalgae including, but not limited to, Chlorella and Chlamydomonas using LiCl under salinity stress and z-vad-fmk under UV stress for inducing autophagic activity for extended periods of time. Also, induction of autophagy by cloning the Chlorella Atg1 gene into Chlamydomonas and short-term induction of autophagy by UV treatment are also shown. The increase in number of lysosomes due to increased autophagic activity of the cells in which autophagy was induced was also shown using FACS. As discussed in the background of the invention, such microalgae, under natural conditions, are known to produce products of commercial interest and inducing autophagy in such microalgae under stress, especially autophagy for extended periods of time, yields high biomass and lipid content, lipids, a variety of biofuel feedstocks, storage starch, triacylglycerols, pharmaceutically useful products, nutraceutically useful products, omega fatty acids etc. The processes disclosed in the present invention for inducing autophagy, and enhancing autophagy by extending the microalgal autophagic activity for longer periods of time where microalgae is exposed to stress, are significant for obtaining economically useful products on a commercial scale.

What has been described and illustrated herein are preferred embodiments of the invention along with some of their variations. The terms and descriptions used herein are set forth by way of illustration only and are not meant as limitations. Those skilled in the art will recognize that many variations are possible within the spirit and scope of the invention, which is intended to be defined by the claims that follow—and their equivalents—in which all terms are meant in their broadest reasonable sense unless otherwise indicated.

1. A method of increasing biomass and lipid content in a micro-organism exposed to stress comprising:
a. cloning in a vector an exogenous gene sequence selected from the group comprising Atg1 gene, Atg6 gene, and Atg8 gene sequence wherein the sequence is at least 50% homologous with Atg1 gene, Atg6 gene, and Atg8 gene, codon optimized for said micro-organism, for inducing autophagy;
b. introducing the vector containing the gene into the genome of the micro-organism to yield a genetically modified micro-organism; and
c. growing the genetically modified micro-organism in suitable medium.

2. The method as claimed in claim 1 wherein, the genetically modified micro-organism is exposed to abiotic stresses comprisinq ultraviolet radiation (UV), salinity, light, unfavourable temperature, alkalinity, nutrient limitation, oxidative stress, senescence, sulfur deficiency, carbon deficiency, nitrogen use inefficiency, or stress due to biotic reasons comprising virus, bacteria, fungus or other stress causing pathogens.

3. The method as claimed in claim 1 wherein, the vector is pChlamy_1.

4. The method as claimed in claim 1 wherein, the exogenous gene has at least 52% homology with Atg1 gene of yeast.

5. The method as claimed in claim 4 wherein, the exogenous gene having at least 52% homology with Atg1 gene of yeast is obtained from Chlorella.

6. A method of increasing biomass and lipid content in a micro-organism exposed to stress, comprising treating the micro-organism with an autophagy inducing agent.

7. The method as claimed in claim 6 wherein, the stress is abiotic stresses comprising ultraviolet radiation (UV), salinity, light, unfavourable temperature, alkalinity, nutrient limitation, oxidative stress, senescence, sulfur deficiency, carbon deficiency, nitrogen use inefficiency, or stress due to biotic reasons comprising virus, bacteria, fungus or other stress causing pathogens.

8. The method as claimed in claim 6 wherein, the UV exposure is not more than 6 hours.

9. The method as claimed in claim 6 wherein, the autophagy inducing agent is z-vad-fmk when the stress is UV.

10. The method as claimed in claim 6 wherein, the micro-organism is treated with 1 mM to 1M of z-vad-fmk for 1 minute to 5 days.

11. The method as claimed in claim 6 wherein, the micro-organism is kept in the dark for 24 hours after UV exposure followed by exposure to light.

12. The method as claimed in claim 6 wherein, salinity exposure is not more than 10 days.

13. The method as claimed in claim 6 wherein, the autophagy inducing agent is LiCl when the stress is salinity.

14. A genetically modified micro-organism exhibiting enhanced autophagy, the micro-organism comprising a vector carrying an exogenous gene sequence selected from the group comprising Atg1 gene, Atg6 gene, and Atg8 gene sequence wherein the sequence is at least 50% homologous with Atg1 gene, Atg6 gene, and Atg8 gene codon optimized for algae, known to induce autophagy.

15. The micro-organism as claimed in claim 14 wherein, the vector is pChlamy_1.

16. The micro-organism as claimed in claim 14 wherein, the exogenous gene has at least 52% homology with Atg1 gene of yeast.

17. The micro-organism as claimed in claim 14 wherein, the exogenous gene having at least 52% homology with Atg1 gene of yeast is obtained from Chlorella.

18. A genetically modified eukaryotic micro-organism exhibiting enhanced autophagy comprising a nucleic acid sequence of SEQ ID No. 1.

19. A genetically modified micro-organism exhibiting enhanced autophagy comprising a nucleic acid sequence coding a protein kinase domain of SEQ ID No. 2.

20. The genetically modified micro-organism as claimed in claim 18 is a photosynthetic micro-organism.

21. A nucleic acid sequence comprising SEQ ID No. 1.

22. A nucleic acid sequence encoding a polypeptide comprising an amino acid sequence of SEQ ID No. 2

23. A polypeptide comprising an amino acid sequence of SEQ ID No. 2

24. A vector comprising a regulatory nucleic acid segment operably coupled to a nucleic acid sequence of SEQ ID No. 1.

25. A vector comprising a regulatory nucleic acid segment operably coupled to a nucleic acid sequence encoding a polypeptide comprising an amino acid sequence of SEQ ID No. 2.
